(19) United States
(12) Patent Application Publication
(10) Pub. No.: US 2011/0296542 A1
(43) Pub. Date: Dec. 1, 2011

(54) EXOGENOUS MATRIX-SUPPORTED TOPICAL APPLICATION OF STEM CELLS TO ORGAN SURFACE

(76) Inventors: Kevin Ka-wang Wang, Gainesville, FL (US); Lam Ping Kuen, Hong Kong (CN); Ng Chi Fai, Hong Kong (CN); Ng Siu Man Simon, Hong Kong (CN); Mak Wing Chung Tony, Hong Kong (CN); Poon Wai Sang, Hong Kong (CN); Lai Bo San Paul, Hong Kong (CN)

(21) Appl. No.: 13/149,186

(22) Filed: May 31, 2011

Related U.S. Application Data
(60) Provisional application No. 61/349,460, filed on May 28, 2010.

(51) Int. Cl. A01K 67/027; A61P 43/00; A61K 35/44; A61K 35/12; A61K 35/14

(52) U.S. Cl. 800/8; 424/93.7

(57) ABSTRACT

A process of tissue regeneration is provided that includes the administration of a stem cell topically to a surface of a tissue. The tissue being in vivo in a subject or in vitro. The tissue has a biocompatible matrix applied around the stem cell and in contact with the surface. After sufficient time, the stem cell infiltrates and regenerates the tissue. A composition is also provided that includes an injured or diseased tissue with a plurality of stem cells proximal to the tissue surface in a biocompatible. A process of increasing a level of cytokines, chemokines, growth factors, anti-apoptotic or anti-necrotic factors in a tissue is provided that includes the administration of a stem cell topically to a surface of a tissue in a biocompatible matrix. After sufficient time, the stem cell releases soluble pro-regenerative factors to increase the levels in the tissue.

### CROSS REFERENCE TO RELATED APPLICATIONS

This application claims priority to U.S. Provisional Application No. 61/349,460 filed May 28, 2010; the contents of which is incorporated herein by reference.

### FIELD OF THE INVENTION

The present invention in general relates to tissue regeneration; and in particular to materials and processes are provided for facilitating regeneration of diseased or injured tissue by administration of stem cells directly to the site of tissue damage and using an adhesive matrix simultaneously to promote i) stem cells adherence to the recipient organ surface, ii) stem cell survival, iii) proliferation, iv) migration, iv) differentiation and v) integration of transplanted stem cells into the organs concerned.

### BACKGROUND OF THE INVENTION

Enhancing cellular regrowth and reconnection is a viable strategy to treat tissue injury arising from degenerative injuries or traumatic impact events such as traumatic brain injury (TBI), muscle oxygen starvation such as during myocardial infarction, and liver or kidney injury. Cellular regrowth mediated tissue repair is especially critical for military personnel who suffered from brain or other injury so they can be returned to active duty more quickly or at least to lead a normal post-military life.

Stem cells represent one possible tool for promoting tissue repair. Adult stem cells are present in a large number of tissues, illustratively bone marrow, blood, liver, muscle, the nervous system, and in adipose tissue. Adult stem cells possess impressive cell plasticity—the ability to differentiate into tissues other than those for which it was believed they were destined. These properties of adult stem cells make them viable sources for therapeutic applications.

Mesenchymal stem cells (MSC) are multipotent cells found in bone marrow and periosteum, and are capable of differentiating into various mesenchymal or connective tissues. For example, such bone-marrow derived stem cells can be induced to develop into myocytes upon exposure to agents such as 5-azacytidine (Wakitani et al., Muscle Nerve, 18 (12), 1417-26 (1995)). It has been suggested that such cells are useful for repair of tissues such as cartilage, fat, and bone (see, e.g., U.S. Pat. Nos. 5,908,784, 5,906,934, 5,827,740, 5,827,735), and that they also have applications through genetic modification (see, e.g., U.S. Pat. No. 5,591,625). While the identification of such cells has led to advances in tissue regrowth and differentiation, the use of such cells is hampered by several technical hurdles. One drawback to the use of such cells is that they are very rare (representing as few as 1/2,000,000 cells), making any process for obtaining and isolating them difficult and costly. Of course, bone marrow harvest is universally painful to the donor. Moreover, such cells are difficult to culture without inducing differentiation, unless specifically screened sera lots are used, adding further cost and labor to the use of such stem cells.

In contrast to the difficulties of isolating MSCs from bone marrow, increasing evidence indicates that MSCs isolated from adipose tissue are easily accessible and possess a higher proliferative rate than those harvested from bone marrow. Previous studies demonstrate that in the presence of beta-mercaptoethanol and dimethylsulphoxide, MSCs can be differentiated into cell lineages expressing neuron specific proteins such as neuron specific enolase (NSE) and neurofilament.

Transplantation of MSC has shown considerable therapeutic potential in a number of animal models of central nervous system disorders such as ischemic brain and spinal cord injury and Parkinson's disease as the transplanted MSC appeared to have the capacity to differentiate into both neuron and glial cell types replacing dysfunctional apoptotic neural cells thereby exerting beneficial effects on functional outcome. Yet, conventional methods of transplantation of stem cells such as through intra-arterial/intravenous injections are inefficient in reaching the target tissue. Direct implantation/injection into a target organ is risky and invasive, such processes include direct intracerebral implantation/injection into a brain lesion. Prior art attempts repair central nervous system damage have also resorted to excision or otherwise damaging a tissue surface prior to direct injection of MSC thereby leading to further complications.

Systemic delivery of MSCs through arterial or venous system is an appealing strategy because by selective delivery through cannulation of specific blood vessels, large amount of cells can be transplanted by infusion. However, when stem cells are injected into the blood and circulated most of them are detained in other internal organs thereby limiting the therapeutic value of such an approach to treatment. Only a small portion of the transplanted cells that remain in systemic circulation eventually pass through the blood-brain barrier and home in on the brain lesion site such as for treatment of TBI. Thus, the therapeutic dose of the infused MSC that reach its target is very minimal. Moreover, there is a threat of arterial embolism or occlusion during intra-arterial administration of MSCs. Another alternate approach is by the direct implantation of cells into target organs using Hamilton syringes. Although the stem cell can be directly injected at the intracerebral lesion site, it is an invasive procedure thus the injection volume and the cell quantity must be minimized. Unfortunately, this approach may pose certain risks of bleeding and also the trituration of cell during the preparation of cell suspension may lead to inevitably damage of the cell membranes and subsequently may adversely affect the viability of the MSCs. In either case in animal studies, before MSC can be injected, trituration through decreasing gauge needles must be done to reduce cell clumps. However, repeated trituration causes unavoidable cell membrane damage that adversely affects MSC viability and subsequent engraftment.

Thus, there exists a need for methods of isolating, expanding, and applying adult stem cells to a site of injury to promote and enhance tissue repair.

### SUMMARY OF THE INVENTION

A process of tissue regeneration is provided that includes the administration of a stem cell topically to a surface of a tissue. The tissue being in vivo in a subject or in vitro. The tissue has a biocompatible matrix applied around the stem cell and in contact with the surface. After sufficient time, the stem cell infiltrates and regenerates the tissue.

A composition is also provided that includes an injured or diseased tissue with a plurality of stem cells proximal to the tissue surface. A biocompatible matrix in simultaneous contact with an intact surface of the tissue is provided.

A process of increasing a level of cytokines, chemokines, growth factors, anti-apoptotic or anti-necrotic factors in an injured or diseased tissue is provided that includes the administration of a stem cell topically to a surface of a tissue. A biocompatible matrix is applied around the stem cell and in contact with the surface. After sufficient time, the stem cell releases soluble pro-regenerative factors to increase the level of cytokines, chemokines, growth factors, anti-apoptotic or anti-necrotic factors in the tissue.

### BRIEF DESCRIPTION OF THE DRAWINGS

FIG. 1 represents a schematic of one embodiment of the inventive process;

FIG. 2 represents the high viability of topically applied stem cells with fibrin glue on injured liver tissue;

FIG. 3 represents the high viability of topically applied stem cells with fibrin glue on injured renal tissue;

FIG. 4 represents the high viability of topically applied stem cells with fibrin glue on injured neural tissue.

FIG. 5 represents the homing of Adipose tissue-derived mesenchymal stem cells into the kidney, liver and small intestine.

### DETAILED DESCRIPTION OF THE PREFERRED EMBODIMENTS

The present invention has utility in the application of stem cells to somatic organs. MSCs exert a therapeutic effect primarily via the release of these soluble pro-regenerative factors that act on local and distant target tissues, instead of, or in addition to differentiation and integration into impaired organs. Topical application offers an efficient delivery of most of the transplanted MSCs to the target organs. Moreover, the concern of MSCs retention by other normal organs, identified in conventional methods is limited. Topical application allows multiple MSCs transplantation without causing major trauma to the target organs and related complications. The further application of MSCs topically on the surface of somatic organs combined with the use of fibrin as an exogenous matrix to keep the MSCs in place on the recipient surfaces also represents a novel solution for the desired result of stem cell delivery to a somatic tissue. MSCs are directly applied according to the present invention to the injured organs in a single topical transplantation. Consequently, a large dosage of paracrine mediators released from the MSCs are continuously exposed to the recipient organs.

The following description of the preferred embodiment(s) is merely exemplary in nature and is in no way intended to limit the scope of the invention, its application, or uses, which may, of course, vary. The invention is described with relation to the non-limiting definitions and terminology included herein. These definitions and terminology are not designed to function as a limitation on the scope or practice of the invention but are presented for illustrative and descriptive purposes only.

Compositions and processes are provided for the treatment, amelioration, recovery, or regeneration of tissues and organs. The terms tissue and organs used synonymously herein. An inventive process illustratively includes isolating stem cells from a patient or another subject, and administering the stem cells to the patient at the site of injury. The present invention is operative with autologous stem cells, exogenous stem cells, or allogenic stem cells. Thus, the invention has utility as a therapeutic treatment for tissue and organ injury, regardless of the causation of physical damage to tissue cells.

The present invention uses exogenous, biocompatible, bio-degradable matrix materials to facilitate cell inoculation on the surface of diseased solid organs to enhance cell migration deeper into the organ structure. The invention also optionally affords simulatenous or sequential delivery of therapeutically relevant cytokines, chemokines, growth factors, proteolytic enzymes and related substances including bioengineered genetic materials directly to the organs from the transplanted stem cells so as to expedite the tissue regeneration. The inventive processes and materials provide for localized administration of stem cells or other desired cells, tissues, proteins, or other modulators to the site of injury or disease. The inventive process provides localization of stem cells to the target area with limited contamination of cells to adjacent areas.

The inventive processes capitalize on the natural differentiable processes present in tissues. Tissues regenerated by the present invention include at least brain, kidney, liver, spinal cord, heart, pancreas, skeletal muscle, lung, large intestine, small intestine, stomach, testis, ovary, bone, peripheral nerve, cartilage, soft tissue, or combinations thereof The inventive methods secure the stem cells on an organ or tissue surface by providing matrix support. This approach enhances the survival and viability of transplanted stem cells and minimizes the trapping of stem cells in other normal or non-diseased or injured organs.

An inventive process illustratively includes extracting a stem cell from a subject by conventional means. A stem cell has the capacity for further differentiation into one or more cell types such as a differentiated cell. A differentiated cell is a cell that possesses one or more characteristics of a somatic cell as found in a subject. The term differentiated cell is not required to mean a terminally differentiated cell although a terminally differentiated cell is similarly envisioned. In some embodiments, a differentiated cell is a precursor cell optionally with one or more phenotypic characteristics of a terminally differentiated cell.

A stem cell is optionally an adult stem cell, fetal stem cell, embryonic stem cell, any stem cell which serves as a delivery vehicle for pharmaceutical drugs or gene therapy, or stem cells having the capacity of homing to sites of injury.

A stem cell is illustratively extracted from bone marrow, blood, or adipose tissue. Optionally, one or more stem cells are extracted from adipose tissue and are as such adipose-derived stem cells. “Adipose-derived stem cells” refer to cells that are extracted from adipose tissue. “Adipose” is any fat tissue. The adipose tissue is optionally brown or white adipose tissue, derived from subcutaneous, omental/visceral, mammary, gonadal, or other adipose tissue site. The adipose is optionally subcutaneous white adipose tissue. Such cells illustratively include a primary cell culture or an immortalized cell line. The adipose tissue is optionally from any organism having fat tissue. Optionally, the adipose tissue is mammalian, such human adipose tissue. A convenient source of adipose tissue is from liposuction surgery, however, the source of adipose tissue or the method of extraction of adipose tissue is not critical to the invention.

A stem cell is optionally a mesenchymal stem cell. The use of mesenchymal stem cells allows isolation from an adult subject and subsequent administration to the adult from which it is isolated, or another adult.

A stem cell is optionally isolated. The terms “purified” or “isolated” as used herein, are intended to refer to a cell, sequesterable from other biological components, wherein the cell is isolated to any degree relative to its naturally-obtainable state, i.e., in this case, relative to its purity within adipose tissue or bone marrow (or umbilical cord) from which it is extracted. A purified cell therefore also refers to a cell free from the environment in which it may naturally occur.

The adipose tissue-derived stem cells of the invention are optionally isolated by a variety of methods known to those skilled in the art illustratively as described in WO 00/53795, the contents of which are incorporated herein by reference. Adipose tissue is optionally isolated from a mammalian subject. Adipose tissue sources illustratively include omental adipose or subcutaneous adipose. In humans, the adipose is optionally extracted by liposuction. If the cells of the invention are to be transplanted into a human subject, the adipose tissue is optionally isolated from that same subject to provide for an autologous transplant. Alternatively, the transplanted cells are allogeneic.

Many techniques are known to those of ordinary skill in the art which can be used to help isolate, culture, induce differentiation, and to characterize the cells of the invention (Gorio et al., 2004, Neuroscience, 125:179-189; Yamashita et al., 2005, J. Cell Sci., 118:665-672; Conley et al., 2004, The International Journal of Biochemistry and Cell Biology, 36:555-567; Kindler, 2005, Journal of Leukocyte Biology, 78:836-844; Fuchs et al., 2004, Cell, 116:769-778; Campos, 2004, Journal of Neuroscience Research, 78:761-769; Dontu et al., 2005, Journal of Mammary Gland Biology and Neoplasia, 10:75-86). The contents of each of these references are incorporated herein in their entirety. Illustratively, stem cells are isolatable from blood by techniques described by Seligman J, et al, Stem Cells Dev., 2009 November; 18(9):1263-71, the contents of which are incorporated herein by reference. Isolating stem cells from bone marrow is illustratively performed by techniques described by Juopperi T A, et al, Exp Hematol., 2007 February; 35(2):335-41.

The term “adult” is defined herein as any non-embryonic or non-juvenile subject. For example the term “adult adipose tissue stem cell refers to an adipose stem cell other than that obtained from an embryo or juvenile subject.

A subject as defined herein is illustratively a mammal. A subject is illustratively a primate including higher primates including humans, and lower primates. Optionally, a subject is a rodent. A rodent illustratively includes a rat or mouse. Other subjects illustratively include a hamster, guinea pig, pig, horse, sheep, bovine, donkey, dog, or cat. A subject is optionally an organism suspected of or having an injury. A subject is optionally a patient.

Administering a stem cell to a patient is illustratively by topical administration, systemic administration, or other localized delivery method. Cell or stem cell administration is optionally administered directly to the surface of a target tissue or organ by injection of cells.

An organ or tissue target of the invention is illustratively diseased or injured. The terms diseased or injured refer to a condition whereby the tissue is abnormal as the result of infection, age, physical injury such as percussive injury or incisive injury, chemical damage, cancer, degeneration, or other condition. Illustrative examples include but are not limited to: myelin involving diseases such as multiple sclerosis; stroke; amyotrophic lateral sclerosis (ALS); chemotherapy; cancer; Parkinson's disease; nerve conduction abnormalities stemming from chemical or physiological abnormalities such as ulnar neuritis and carpel tunnel syndrome; other peripheral neuropathies illustratively including sciatic nerve crush (traumatic neuropathy), streptozotozin (STZ) (diabetic neuropathy), and antimitotic-induced neuropathies (chemotherapy-induced neuropathy); experimental autoimmune encephalomyelitis (EAE); delayed-type hypersensitivity (DTH); rheumatoid arthritis; epilepsy; pain such as neuropathic pain; kidney disease; liver disease; and intra-uterine trauma.

An inventive process optionally includes applying a matrix prior to, along with, or subsequent to administration of a stem cell. A matrix is optionally an adhesive, tacky to hold cells in position on the recipient organ surface whether liquid, semi-solid, solid or of some other glue-like physical. A matrix optionally includes modulators of cell proliferation, migration or activity. A matrix also optionally includes enzymes that promote matrix adhesion to a tissue, matrix integrity, or temporally mediated matrix degradation or absorption.

A matrix illustratively includes fibrin, fibronectin, gelatinous protein mixtures or other man-made mixtures, collagen, laminin, other biocompatible matrix materials, derivatives thereof, or combinations thereof. In some embodiments a matrix is a sprayable, spreadable, or layerable fibrin glue. Due to its haemostatic and sealing properties, the FDA approved (1998) fibrin glue (also refereed as fibrin sealant or tissue adhesive). Sources of or production of fibrin glues are known in the art. Optionally, fibrin glue is produced form a patient's own fibrinogen and thrombin such as by the method of Lendeckel, S, et al., Journal of Cranio-Maxillofacial Surgery, 2004; 32, 370-373. Fibrin glue improves the adherence of split-thickness skin grafts to recipient burn wounds and thus increases the graft take rate. It has also been used as a delivery carrier for cultured keratinocytes. The cells are pre-mixed with fibrin, a component of fibrin glue, and then sprayed onto the wounds to facilitate their re-epithelization. In vitro studies show that human keratinocytes remain viable in suspension in fibrin for at least five days. Some human skin substitutes make use of the matrix of fibrin glue to proliferate cultured fibroblasts, keratinocytes or a combination of both. Fibrin glue has also been extended to bone tissue regeneration. The fibrin-stabilizing factor XIII of fibrin glue favors migration of undifferentiated MSC on the highly cross-linked matrix and facilitates cellular proliferation and differentiation into estrogenic lineage.

An inventive process illustratively includes mixing stems cells, optionally along with other cells, reagents, or modulators, with fibrinogen, producing a cell containing fibrin glue and topically administering the fibrin glue to a tissue or organ of a subject. An inventive process also illustratively includes topically administering a stem cell to a target tissue or organ and over layering the stem cell with a matrix, optionally fibrin glue. The word “topically” as used herein is administration to the surface of a target tissue, organ, or cell. Illustratively, topical administration is overlaying, spraying, or dropping cells onto a target.

An inventive process also illustratively includes altering the level of one or more cytokines, chemokines, growth factors, anti-apoptotic or anti-necrotic factors, and related paracrine mediators in a target tissue or organ following administration of one or more stem cells. Cytokines are optionally chemokines. Illustrative examples of cytokines, chemokines, growth factors, anti-apoptotic or anti-necrotic factors, and related paracrine mediators include C5a, CD40 Ligand, G-CSF, GM-CSF, GROα, I-309, sICAM-1, IFN-γ, IL-1α, IL-1β, IL-1ra, IL-2, IL-4, IL-5, IL-6, IL-8, IL-10, IL-12p70, IL-13, IL-16, IL-17, IL-17E, IL-23, IL-27, IL-32α, IP-10, I-TAC, MCP-1, MIF, MIP-1α, MIP-1β, Serpin E1, RANTES, SDF-1, TNFα, TGFβ₁, VEGF, MCP-1, and sTREM-1, among others. Anti-apoptotic factors are also illustratively described by Rehman J, et al, Circulation. 2004 Mar. 16; 109(10):1292-8, the contents of which are incorporated herein by reference. Anti-necrotic factors also illustratively include IL-6. The altered level of cytokines compared either to the levels prior to therapy, or to an expected normal or abnormal value is indicative of stem cell infiltration or stem cell related cell infiltration into a target tissue or organ.

Techniques for measuring the levels of cytokines, chemokines, growth factors, anti-apoptotic or anti-necrotic factors, and related paracrine mediators in a tissue are known in the art. Illustratively examples include ELISA, flow cytometry, mass spectroscopy, western blot, liquid chromatography, combinations thereof, or other techniques and combinations of techniques known in the art. Reagents for performing these techniques are similarly known in the art as is where to obtain such reagents. Illustratively, the human cytokine array panel PROTEOME PROFILER from R&D Systems, Santa Cruz, Calif. is operable to analyze cytokines from tissue. Other assays for growth factors are available from GE Healthcare Bio-Sciences Corp., Piscataway, N.J.

Also included is a kit for promoting tissue repair including a stem cell and a biocompatible matrix. A kit illustratively includes a stem cell. The stem cell is optionally isolated from a subject. A biocompatible adhesive as described herein is also included in an inventive kit. A stem cell is illustratively an adipose tissue derived stem cell. Illustratively, a mesenchymal stem cell is included.

Various aspects of the present invention are illustrated by the following non-limiting examples. The examples are for illustrative purposes and are not a limitation on any practice of the present invention. It will be understood that variations and modifications can be made without departing from the spirit and scope of the invention. While the examples are generally directed to mammalian tissue, specifically, analyses of murine tissue, a person having ordinary skill in the art recognizes that similar techniques and other techniques known in the art readily translate the examples to other organisms such as humans. Reagents illustrated herein are commonly cross reactive between mammalian species or alternative reagents with similar properties are commercially available, and a person of ordinary skill in the art readily understands where such reagents may be obtained. It is further understood that while the examples are directed to mesenchymal stem cells that one of ordinary skill in the art readily understands variations of these techniques useful to other types of stem cells. Finally, one of ordinary skill in the art similarly understands that the fibrin glue used in the following examples is substitutable by other matrices as described herein or known in the art.

### EXAMPLE 1

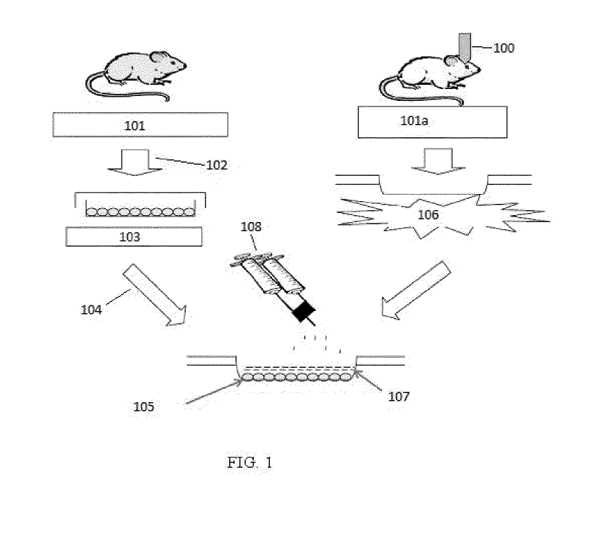

Isolation, culture, and characterization of adipose-tissue derived mesenchymal stem cells (ADMSC). Abdominal subcutaneous adipose tissue is taken from transgenic green fluorescent protein (GFP) Sprague-Dawley rats (SD-Tg (CAG-EGFP) CZ-0040sb, 12-week-old males) (see FIG. 1). The tissues are minced, digested with type I collagenase (Sigma-Aldrich, St. Louis, Mo.) for 30 minutes and then passed through a 100 μm filter to discard the cell debris. The cell pellets are suspended in DMEM containing 10% FBS, 100U penicillin/100 μg streptomycin/0.25 μg fungizone and cultured at 37° C. and 5% CO₂ in humidified incubator. Techniques are essentially as described by Andreas S, and Christa B., Stem Cells, 2007; 25: 818-827; Mothe A J, et al., J Histochemistry & Cytochemistry, 2005; 53: 1215-1226; and Tao W, et al., Stem Cells, 2007; 25: 670-678, the contents of each of which are incorporated herein by reference.

The phenotype of ADMSCs is determined by flow cytometry (Becton Dickinson) using phycoerythrin (PE)-conjugated antibodies against CD90 and CD45. The adipogenic, estrogenic and chondrogenic differentiation potential of the ADMSCs are tested essentially as described by Buhring H J, et al., Ann NY Acad Sci, 2007; 1106: 262-271, and Yusuke S, et al., Arthritis & Rheumatism, 2005; 52: 2521-2529.

### EXAMPLE 2

MSCs are first derived from the subcutaneous adipose tissue of transgenic Sprague-Dawley (SD) rats expressing green fluorescent protein (GFP). The MSCs express CD90 (79%), but not CD45, and are capable of in vitro adipogenic, chrondrogenic and osteogenic differentiation under selective culture conditions. To stimulate the mobilization of the topically applied MSCs, experimental models of severe ischemia-reperfusion injury (IRI) of kidney, liver and small intestine are induced in test wild-type SD rats (N=19) by occlusion of kidney pedicle for 40 minutes (3); clamping portal vein and hepatic artery and bile duct for 30 minutes (4); and occlusion of main vascular pedicle, together with ligation of collateral vessels to the bowel segment for 90 minutes (5).

Two days after the IRI, 7×106 GFP-MSCs at passage 2-3 suspended in 200 μl phosphate buffer saline are applied directly to the organ surfaces of the ischemic kidney, liver and small intestine of the test animals (N=9). A thin layer of fibrin glue (Tisseel®, Baxter Healthcare S.A., Wallisellen, Switzerland) is then applied to the recipient surfaces. In the control groups, either MSCs alone are applied to the ischemic organs (N=6) or MSCs together with fibrin glue are applied to the non-ischemic organs (N=2). The trafficking of GFP-MSCs is then examined by immunohistochemistry staining on serial paraffin sections with rabbit polyclonal anti-GFP (1:4000, Abcam Inc., Cambridge, UK).

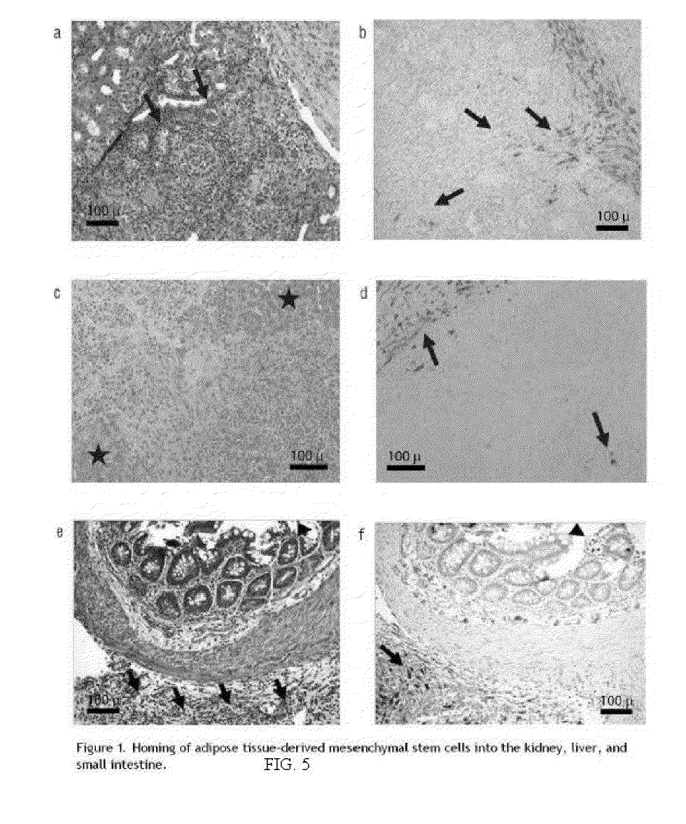

FIG. 5 shows Homing of Adipose tissue-derived mesenchymal stem cells into the kidney, liver and small intestine. (A) Significant inflammatory cells (arrows) are seen infiltrating into the renal cortex as a subcapsular wedge-shaped scar, characterized by tubular atrophy with interstitial fibrosis. (B) MSCs as indicated by anti-GFP antibody (anti-GFP, Abcam inc., Cambridge, UK) are shown to migrate into injured renal cortical parenchyma 3 days after cell transplant. Magnifications ×100. (C) Injured liver parenchyma is replaced by fibrovascular tissue with inflammatory cells infiltrate and extravasated red blood cells. Residual hepatic lobules are indicated by star. (D) MSCs as indicated by anti-GFP antibody (anti-GFP, Abcam inc., Cambridge, UK) are seen to migrate into the injured site of the liver 5 days after cell transplant (arrows). Magnifications ×200. (E) Fibroblastic proliferation, associated with moderate inflammation, is noted on the serosal surface of the injured site of the small intestine (arrows), where MSCs were topically applied. Sloughing of epithelium at the tips of villous (arrow-head) and the uneven muscularis propria marked the ischemic damaged site of the small intestine. (F) Viable GFP labelled MSCs (anti-GFP, Abcam inc., Cambridge, UK) are seen amongst the fibroblastic proliferation on the serosal surface (arrows) and some MSCs have migrated into lamina propria of the injured small intestine 7 days after cell transplant (arrow head). Magnifications ×200.

Mobilization of MSCs and their subsequent homing to injured tissues is mediated by various chemokines and their receptors. As the MSCs are topically applied to the organs two days after the ischemic reperfusion injury, the homed MSCs are exposed to a hostile microenviroment with hypoxia and the infiltrative activated neutrophils. Due to its haemostatic and sealing properties, fibrin glue is widely used in many areas of surgery. It also acts as a delivery carrier for cultured keratinocytes. Similar results are obtainable with ischemically and trauma injured brain lesions, as well as peripheral nerve tissues.

### EXAMPLE 3

Rat traumatic brain injury model: A controlled cortical impact (CCI) device is used to produce model of traumatic brain injury (TBI) in wild-type SD rats essentially as described by Prins M L, et al., J Neurosci Res, 2005; 82(3):413-20; and Ringger, N. C., et al., J. Neurotrauma, 2005; 21, 1443-1456, the contents of each of which are incorporated herein by reference. CCI is used to generate injured brain tissue and CSF samples. Adult male (280-300 g) Sprague-Dawley rats (Harlan) are anesthetized with 4% isoflurane in a carrier gas of 1:1 O₂/N₂O (4 min) followed by maintenance anesthesia of 2.5% isoflurane in the same carrier gas. Core body temperature is monitored and maintained at 37° C. Animals are mounted in a stereotactic frame and a unilateral craniotomy (7 mm diameter) is performed adjacent to the central suture, midway between bregma and lambda. The dura mater is kept intact over the cortex. Brain trauma is produced using a Benchmark™ Stereotaxic Impactor (MyNeurolab) by impacting the left cortex (ipsilateral cortex) with a 4 mm diameter impactor tip at a velocity of 3.5 m/s, 2.5 mm compression depth and a 200 ms dwell time (compression duration). Brain trauma is produced using a Benchmark™ Stereotaxic Impactor (MyNeurolab) by impacting the left cortex (ipsilateral cortex) with a 4 mm diameter impactor tip at a velocity of 3.5 m/s, 2.5 mm compression depth and a 200 ms dwell time (compression duration). GFP-MSCs are applied onto the TBI site. Sham-injured control animals undergo identical surgical procedures but do not receive an impact injury.

### EXAMPLE 4

Ischemia-reperfusion injury (IRI) animal models. Under anesthesia, ischemia-reperfusion injury (IRI) in liver is induced by clamping portal vein and hepatic artery with a bulldog clamps for 30 minutes and then releasing the clamp. Ischemia-reperfusion injury in brain as severe incomplete ischemia is induced by bilateral clamping of common carotid arteries for 45 minutes using micro vessel clips, then releasing the clips. Ischemia-reperfusion injury in kidney is induced by-bilateral occlusion for 2 hours using bulldog to clamp renal pedicles (both renal artery and renal vein) then releasing the clamps.

### EXAMPLE 5

Exogenous matrix-supported stem cells are applied to an organ surface and secured by application of fibrin glue to provide matrix support. GFP-rats provide mesenchymal stem cells for transplantation into normal (wild-type) rats as schematically depicted in FIG. 1. For example, the cells and matrix matter are applied to the surface of recipient injured organs. Examples of recipient injured organs include traumatically (CCI) injured brain, or brain, liver or kidney subjected to ischemic reperfusion injury. Topical transplantation of concentrated (1-7×10⁶) mesenchymal stem cells (e.g. GFP-adipose tissue derived mesenchymal stem cells from GFP-rats or GFP-ADMSCs) are suspended in 50-200 μl saline and uniformly applied to the surface of recipient injured organs with a small flat- or round-end spatula. A thick layer of viscous matrix material (e.g. fibrin glue) is dropped, sprayed, or overlayed onto the stem cell population or layer (FIG. 1). For example, exogenous fibrin glue (Baxter, Deerfield, Ill.) consisting of aprotinin and thrombin reconstituted in physiological solution with 1 mM calcium chloride is layered onto the cells. The incision wound is closed by suturing after the matrix material (e.g. fibrin glue) solidifies or is stabilized.

### EXAMPLE 6

Immunohistochemistry. The migration of GFP-ADMSCs into target injured tissue is detected by immunohistochemistry of using rabbit monoclonal anti-GFP (Cell Signalling Technologies, Danvers, Mass.) or rabbit polyclonal anti-GFP (Abcam). Liver, brain and kidney tissues are collected from sacrificed animals 3-7 days after ADMSC transplant and fixed in 4% neutral formalin. Antigen unmasking is performed by microwaving in citrate buffer. After blocking in 5% normal goat serum, sections are incubated with rabbit anti-GFP. For the purpose of intrinsic peroxidase elimination, sections are immersed in 3% hydrogen peroxide for 30 minutes. After incubation with biotinylated goat anti-rabbit IgG secondary antibody, the antibody complex is detected using horseradish peroxidase complex (Vector Laboratories Inc. Burlingame, Calif.) and diaminobezidine (DAB). For the isocontrol slides, the primary antibody is replaced by a normal rabbit IgG fraction or normal rabbit serum.

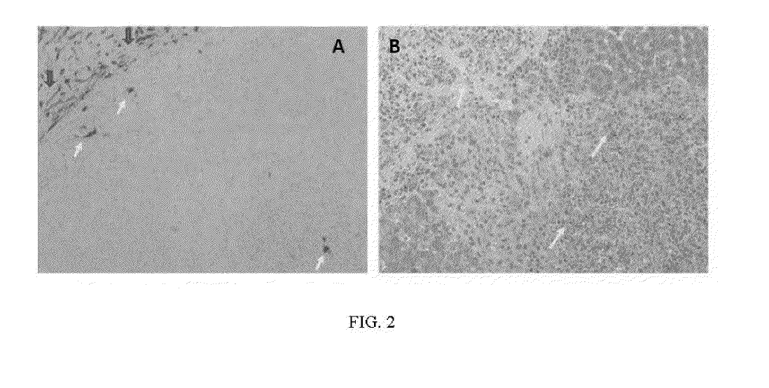

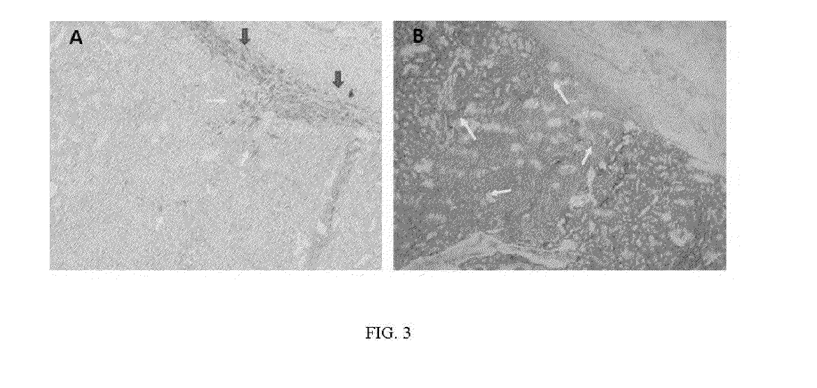

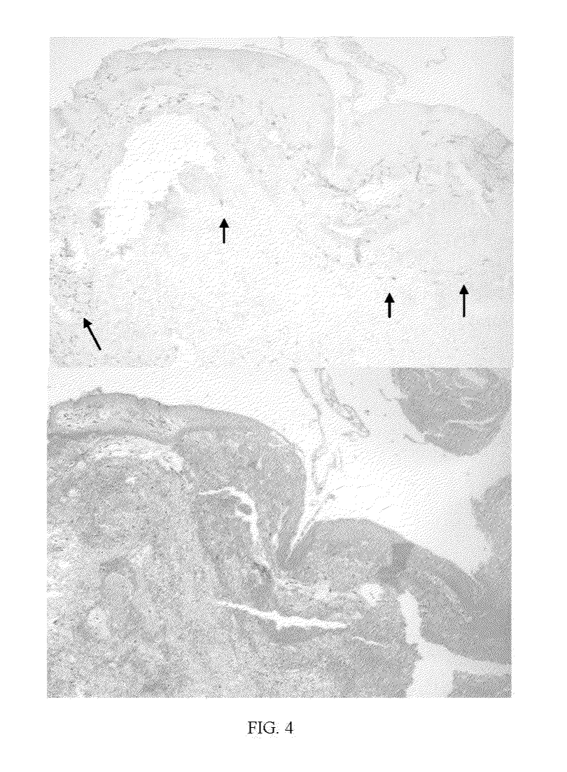

The exogenous fibrin matrix supports the inoculation of GFP-ADMSCs on the recipient target tissue surface. After topical transplantation, the cells migrate into the ischemia regions in liver (FIG. 2) kidney (FIG. 3) and brain (FIG. 4).

FIG. 2 illustrates immunohistochemical staining of topically applied GFP-ADMSCs incorporated into liver parenchyma. Anti-GFP binding illustrates the topically applied mesenchymal stem cells on the liver surface possessing high viability (see blue arrows) (A) and their migration into the liver parenchyma structure 7 days after GFP ADMSC transplant (yellow arrows indicating brown-stained cells (A), and red-stained cells (B)). The blue arrows identify the topically applied GFP-ADMSCs remaining at the boundary of the liver capsule. The cells further migrate into liver parenchyma regions (yellow) with ischemia-reperfusion injury as showed in H&E (hematoxylin and eosin) staining. Rabbit polyclonal anti-GFP (Abcam) is used.

FIG. 3 illustrates immunohistochemical staining of topically applied GFP-ADMSCs incorporated into renal cortex. Anti-GFP binding illustrates the topically applied mesenchymal stem cells on the renal cortex surface with high viability (see blue arrows) and their migration into the deeper cortical area 3 days after GFP MSC transplant (yellow arrows indicating brown-stained cells (A), and red-stained cells (B)). The blue arrows indicate the topically applied GFP-ADMSCs remain at the boundary of the kidney capsule. The cells further migrate into kidney cortex regions (yellow) with ischemia-reperfusion injury as showed in H&E staining.

FIG. 4 shows immunohistochemical staining of topically applied GFP-labeled mesenchymal stem cells (GFP-MSCs) penetrating and integrating into cortical layer of rat brain subjected to cerebral ischemia. (A) Positive GFP staining (arrows) using (Rabbit monoclonal antibody) 5 days after topical application of GFP-MSCs. Magnification ×100. (B) H&E staining. Magnification ×100.

Methods involving conventional biological techniques are described herein. Such techniques are generally known in the art and are described in detail in methodology treatises such as Molecular Cloning: A Laboratory Manual, 2nd ed., vol. 1-3, ed. Sambrook et al., Cold Spring Harbor Laboratory Press, Cold Spring Harbor, N.Y., 1989; and Current Protocols in Molecular Biology, ed. Ausubel et al., Greene Publishing and Wiley-Interscience, New York, 1992 (with periodic updates). Immunological methods (e.g., preparation of antigen-specific antibodies, immunoprecipitation, and immunoblotting) are described, e.g., in Current Protocols in Immunology, ed. Coligan et al., John Wiley & Sons, New York, 1991; and Methods of Immunological Analysis, ed. Masseyeff et al., John Wiley & Sons, New York, 1992.

It is appreciated that all reagents are obtainable by sources known in the art unless otherwise specified. Methods of nucleotide amplification, cell transfection, and protein expression and purification are similarly within the level of skill in the art.

### REFERENCE LIST

Alexander H, Petter P, Wolfgang F, Reinhild K F, Dragan L, Heinz R, Martijn V G. The long-term neurocompatibility of human fibrin sealant and equine collagen as a biomattrices in experimental spinal cord injury. Experimental Toxicologic Path 2007; 58:

Andreas S, Christa B. Conscise review: adipose tissue-derived stromal cells-basic and clinical implications for novel cell-based therapies. Stem Cells 2007; 25: 818-827

Auger F A, Guignard R, Lopez Valle C A, Germain L. Role and innocuity of Tisseel, a tissue glue, in the grafting process and in vivo evolution of human culturd edidermis. Br J Plast Sur 1993; 46: 136-142.

Brody D L, Holtzman D M (2006) Morris water maze search strategy analysis in PDAPP mice before and after experimental traumatic brain injury. Exp Neurol 197:330-340.

Brody D L, Mac D C, Kessens C C, Yuede C, Parsadanian M, Spinner M, Kim E, Schwetye K E, Holtzman D M, Bayly P V (2007) Electromagnetic controlled cortical impact device for precise, graded experimental traumatic brain injury. J Neurotrauma 24:657-673.

Buhring H J, Battula V L, Treml S, Schewe B, Kanz L, Vogel W. Novel markers for prospective isolation of human MSC. Ann NY Acad Sci 2007; 1106: 262-271

Burd, A., Chan, E S Y, Lam, P K. Post burn keloid with keratinocyte cell suspension and fibrin glue. In: Cultured Human Keratinocytes and Tissue Skin substitutes—State of The Art. Edited by Raymund E. Horch, Andrew M. Munster, Brauce Achauer. Published by Thieme 2001. P 298-304

Catherjee P K, Zachrowski K, Cuzzocrea S, et al. Inhbition of poly (ADP-ribose) synthetase reduces renal ischemia-reperfusion injury in the anesthetized rat in vivo. FASEB J 2000; 14: 641-651.

Chamberlain G, Fox James, Ashton B, Middleton J. Concise review: Mesenchymal stem cells: Their phenotype, differentiation capacity, immunological features, and potential for home. Stem Cells 2007; 25: 2739-2749

Clarke D L, Johansson C B, Wilbertz J, Veress B, Nilsson E, Karlstorm H, Lendahl U, Frisen J. Generalized potential of adult neural stem cells. Science 2000: 288: 1660-1663

Corti S, Locatelli F, Papadimitriou D, Strazzer S, Comi G P. Somati stem cell research for neural repair: current evidence and emerging perspectives. J Cell Mol Med 2004; 8: 329-337

Fatar M, Stroick M, Griebe M, marwedel I, et al. Lipoaspirate-derived adult mesenchymal stem cells improve functional outcome during intracerebral haemorrhage by proliferation of endogenous progenitor cells stem cells in intracerebral hemorrhage. Neuroscience letter 2008; 443: 174-178

Gao J, Dennis J, Muzic R, Lundberg M, Caplan A I. The dynamic in vivo distribution of bone marrow-derived mesenchymal stem cells after infusion. Cells Tissues Organ 2001; 169: 12-20.

Hangy, Henderson D, Blackstad M, et al. neuroectodermal differentiation from mouse multipotent adult progenitor cells. Proc Natl Acad Sci USA 2003; 100(suppl 1): 11854-11860

Harting M T, Sloan L E, Jimenez F, Baumgartner J, Cox C S Jr. (2009) Subacute neural stem cell therapy for traumatic brain injury. J Surg Res. 2009 May 15; 153(2):188-94. Epub 2008 Apr. 23.

Haverich A, Walterbush G, BorstHG. The use of fibrin glue for sealing vascular prothesis of high porosity. Thorac Cardiovasc Surg; 29: 252-256

Hermann A, Gastl R, Leibau S, et al. Efficient generation of neural stem cell-like from adult bone marrow stromal cells. J Cell Sci 2004; 17: 4411-4422.

Horwitz E M, Dominici M. How do mesenchymal stromal cells exert their therapeutic benefit? Cytotherapy 2008; 10: 771-774.

Jin K, Sun Y, Xie L, Mao X O, Childs Peel A, et al. Comparison of ischemia-directed migration of neural precursor cells after intrastrial, intravenous, or intravenous transplantation in the rat. Neurobiology Disease 2005; 18: 366-374

Krabbe C, Zimmer J, Meyer M. Neural transdifferentiation of mesenchymal stem cells: A critical review. APMIS 2005; 113: 831-844

Laitakari K, Luotonen J. Autologous and homologous fibrinogen sealants: adhesive strength. Laryngoscope 1989; 99: 974-976.

Lam. P K, Chan, E S Y, Liew, C T, Lau, C H, Yen, S C, King, W W K. Effectiveness of collagen dermis membrane and fibrin on cultured epidermal graft using athymic mice model. Annals of Plastic Surgery 1999; 43: 523-528

Lee O K. Fibrin glue as a vehicle for mesenchymal stem cell delivery in bone regeneration. J Chin Med Yamada Y, Boo J S, Ozawa R, Nagasaka T, Okazaki Y, Hata K I, Ueda M. Bone regeneration following injection of mesenchymal stem cells and fibrin glue with a biodegradable scaffold. J Cranio-Maxillofacial Surg 2003; 31: 27-33

Lee T H, Yoon J Y. Intracerbral transplantation of human adipose tissue stromal cells after middle cerebral artery occlusion in rats. J Clin Neuroscience 2008; 15: 907-912

Lengdeckel S, Jodicke A, Christophis P, Heidinger, Wolff J, et al. Autologous stem cells (adipose) and fibrin used to treat widespread traumatic calvarial defects: case report. 2004; 32: 370-373.

Longhi L, Zanier E R, Royo N, Stocchetti, Mcintosh T K. Stem cell transplantation as a therapeutic stratedy for traumatic brain injury. Transplant Immunology 2005; 15: 143-148

Mahmood A, Lu D, Lu M, et al. Treatment of brain injury in adult rats with intravenous administration of human bone marrow stromal cells. Neurosurgery 2003; 697-702

Mahmood A, Lu D, Qu C, Goussev. Human marrow stromal cell treatment provides long-last benefit after traumatic brain injury in rats. Neurosurgery 2005; 57: 1026-1031

Mahmood U, Weissleder R. Near-infrared optical imaging of proteases in cancer. Molecular Cancer 2003; 2: 489-496

Mattiasson G J, Philps M, Tomasevic G, Johansson B B, Weiloch T, McIntosh T K. The rotating pole test: evaluation of its effectiveness in assessing functional motor deficits following experimental head injury in the rat. J Neuroscience Methods 2000; 95: 75-82

Mooney D J, Vandenburgh H. Cell delivery mechanism for tissue repair. Cell Stem Cell 2008; 2: 205-213

Mothe A J, Kulbatski I, van Bendegem R L, Lee L, et al. Analysis of green fluorescent protein expression in transgenic rats for tracking transplanted neural stem/progenitor cells. J Histochemistry & Cytochemistry 2005; 53: 1215-1226

Ntziachristos V, Weissieder R. In vivo optical imaging of molecular function using NIR fluorescent probes. IN: Fujimoto J, Farkas D. (eds). Biomedical optical imaging, New York: Academic Press, 2003

Pan H C, Yang D Y, Chiu Y T, Lai S Z, Wang Y C, Chang M H, Cheng F C. Enhanced regeneration in injured sciatic nerve by human amniotic mesenchymal stem cell. J Clin Neuroscience 2006; 13: 570-575

Phinney D G., Prockop D J. Concise review: mesenchymal stem/multipotent stromal cells: the state of transdifferentiation and modes of tissue repair-current views. Stem Cells 2007; 25: 2896-2902

Prins M L, Fujima L S, Hovda D A. (2005) Age-dependent reduction of cortical contusion volume by ketones after traumatic brain injury. J Neurosci Res. 2005 Nov. 1; 82(3):413-20.

Quaedackers J S, Beuk R J, Bennet L, et al. An evaluation of methods for grading histologic injury following ischemia/reperfusion of small bowel. Transplantation Proc 2000; 32: 1307-1310

Ringger, N. C., O'Steen, B. E., Brabham, J. G., Silver, X., Pineda, J., Wang, K. K. W. and Hayes, R. L. (2005) A novel marker for traumatic brain injury: CSF αII-spectrin breakdown product levels. J. Neurotrauma 21, 1443-1456.

Ronfard V, Broly H, Mitchell, Galizia J P, Hochart D, Chambon E. et al. Use of human keratinocytes cultured on fibrin glue in the treatment of burn wounds. Burns 1991: 17: 181-184. Meana A, Lglesias J, Rio M D, Larcher F, Madrigal B, Fresno M F, et al. Large sueface of cultured human epithelium obtained on a dermal matrix based on live fibroblast-containing fibrin gel. Burns 1998; 24: 621-630

Sander D, Recta L V, Sandrine F, Arlene K V V, Thomas M V G. IL-10 attenuates hepatic I/R injury and promotes hepatocyte proliferation. J Surg Res 2007; 141: 176-182.

Shear D A, Tate M C, Archer D R, Hoffman S W, Hulce V D, Laplaca M C, Stein D G. 2004. Neural progenitor cell transplants promote long-term functional recovery after traumatic brain injury. Brain Res. 1026:11-22.

Slavin S, Basan G S, Dimitrios Karussis. The potential use of adult stem cells for the treatment of multiple sclerosis and other neurodegenerative disorders. Clin Neurology Neurosugery 2008; 110: 943-946.

Svetlov, S. I., Ignatova, T. N., Wang, K. K. W. Hayes, R. L., Kukekov, V. G. (2004) Growth and Clonal Expansion of Neural Stem/Progenitor Cells by Lysophosphatidic acid (LPA) in Mouse Neurospheres. J. Hematotherapy & Stem Cell Research, 13, 685-693.

Tao W, Evans B G, Yao J et al. Enhanced green fluorescent protein is a nearly ideal long-term expression tracer for haematopoietic stem cells, wheras DsRed-express fluorescent fluorescent protein is not. Stem Cells 2007; 25: 670-678

Walk P A, Shah S K, Harting M T, Cox Jr C S. Progenitor cell therapies for traumatic brain injury: barriers and opportunities in translation. Disease Models & Mechanisms 2009; 2: 23-38

Wang, J., Ding F, Gu Y, Liu J, Gu X. Bone marrow mesenchymal stem cell promote cell proliferation and neutrophic function of Schwann cells in vitro and vivo. Brain Res 2009; 1262; 7-15

Yagi H, Soto-Gutierrez A, Parekkadan B, et al. Mesenchymal stem cells: Mechanisms of immunodulation and homing. Cell Transplant 2010; 19: 667-67.

Yusuke S, Ichiro S, Kazuyoshi Y, Takeshi M. Comparison of human stem cells derived from various mesenchymal tissues. Arthritis & Rheumatism 2005; 52: 2521-2529

Zhu Y, Liu T, Song K, Fan X, Ma X, Cui Z. Adipose-derived stem cells: a better stem cell than BMSC. Cell Biochem Funct 2008: 26: 664-675

Zuk P A, Zhu M, Ashjian P, De Ugarte D A, Huang J I, Mizuno H, et al. Human adipose tissue is a source of multipotent stem cells. Molecular Biology 2002; 13: 4279-4295.

Zulma G, Hadi A, Yossi G, Nadav K, Gadi P, Dan G. Mesenchymal stem cell. Principles of Regenerative Medicine 2008; P318-343

Patents and publications mentioned in the specification are indicative of the levels of those skilled in the art to which the invention pertains. These patents and publications are incorporated herein by reference to the same extent as if each individual application or publication was specifically and individually incorporated herein by reference.

The foregoing description is illustrative of particular embodiments of the invention, but is not meant to be a limitation upon the practice thereof The following claims, including all equivalents thereof, are intended to define the scope of the invention.

1. A process of tissue regeneration comprising:
administering a stem cell topically to a surface of a tissue; and
applying a biocompatible matrix around said stem cell and in contact with the surface; and
allowing sufficient time for said stem cell to infiltrate and regenerate the tissue.

2. The process of claim 1 further comprising isolating said stem cell from a subject receiving said stem cell.

3. The process of claim 1 wherein said stem cell is isolated from are selected from adult stem cells, tissue-specific stem cells, fetal stem cells, cord blood stem cells or cells derived from the umbilical cord, embryonic stem cells, induced pluripotent stem cells, pluripotent mesenchymal stem cells, multipotent mesenchymal stem cells or combinations thereof.

4. The process of claim 1 wherein said stem cell further comprises a plurality of said stem cell and said plurality of said stem cells are pluripotent mesenchymal stem cells or multipotent mesenchymal stem cells.

5. The process of claim 1 wherein said stem cell is isolated from at least one of fetal tissue, fetal organs, adipose tissue, bone marrow or blood.

6. The process of claim 1 wherein said matrix has a tack that holds said stem cell in position on the surface, said matrix comprising fibrin, fibronectin, gelatinous protein, collagen, laminin, polysaccharides, or combinations thereof.

7. The process of claim 6 wherein said matrix comprises as a majority constituent of fibrin.

8. The process of claim 1 wherein the tissue is brain, kidney, liver, spinal cord, heart, pancreas, skeletal muscle, lung, large intestine, small intestine, stomach, testis, ovary, bone, cartilage, periphery nerve, soft tissue or combinations thereof.

9. A composition comprising:
an injured or diseased tissue;
a plurality of stem cells; and
a biocompatible matrix in simultaneous contact with an intact surface of the tissue

10. The composition of claim 9 further comprising said matrix is tacky and said tissue is within a subject.

11. The composition of claim 10 wherein said matrix comprises at least one of fibrin, fibronectin, albumin, collagen, laminin, or combinations thereof.

12. The composition of claim 9 wherein said plurality of stem cells are autologous to the tissue and said matrix comprises fibrin.

13. The composition of claim 12 wherein said matrix further comprises at least one of coagulation factor XIII or nutrient that supports cell proliferation or homeostasis or tissue repair or stem cell homing.

14. A process of increasing a level of cytokines, chemokines, growth factors, anti-apoptotic or anti-necrotic factors in an injured or diseased tissue comprising:
administering a stem cell topically to a surface of a tissue; and
applying a biocompatible matrix around said stem cell and in contact with the surface; and
allowing sufficient time for said stem cell to increase the level of cytokines, chemokines, growth factors, anti-apoptotic or anti-necrotic factors in an injured or diseased tissue.

15. The process of claim 14 further comprising assaying a sample of the tissue for anti-inflammatory factors, tissue repair factors, levels of cytokines, chemokines, growth factors, anti-apoptotic factors, or anti-necrotic factors.

16. The process of claim 14 wherein the tissue is brain, kidney, liver, spinal cord, heart, pancreas, skeletal muscle, lung, large intestine, small intestine, stomach, testis, ovary, bone, cartilage, peripheral nerve, soft tissue or combinations thereof.

17. The process of claim 14 wherein said stem cell is isolated from adult stem cells, tissue-specific stem cells, fetal stem cells, cord blood stem cells, embryonic stem cells, induced pluripotent stem cells, pluripotent mesenchymal stem cells, multipotent mesenchymal stem cells or combinations thereof.

18. The process of claim 17 wherein said stem cell is plurality of said stem cell and said plurality are pluripotent mesenchymal stem cells or multipotent mesenchymal stem cells.

19. The process of claim 17 wherein said stem cell is one of an adult stem cell, fetal stem cell, or embryonic stem cell.

20. The process of claim 14 wherein said stem cell is isolated from at least one of fetal tissue, adipose tissue, bone marrow, or blood.
